(19) United States
(12) Patent Application Publication
(10) Pub. No.: US 2024/0084279 A1
(43) Pub. Date: Mar. 14, 2024

(54) ENGINEERED PROTEASES WITH ENHANCED AUTOLYSIS RESISTANCE

(71) Applicant: Waters Technologies Corporation, Milford, MA (US)

(72) Inventors: Balasubrahmanyam Addepalli, Hopkinton, MA (US); Abraham S. Finny, Marlborough, MA (US); Matthew A. Lauber, North Smithfield, RI (US)

(73) Assignee: Waters Technologies Corporation, Milford, MA (US)

(21) Appl. No.: 18/366,035

(22) Filed: Aug. 7, 2023

Related U.S. Application Data
(60) Provisional application No. 63/395,986, filed on Aug. 8, 2022.

(51) Int. Cl. C12N 9/50

(52) U.S. Cl. CPC C12N 9/50; C12Y 304/21004; C12Y 304/2105; C12Y 304/24033

(57) ABSTRACT

This present disclosure relates to engineered protease enzymes, including trypsin, Lys-C and Asp-N proteases, that have enhanced autolysis resistance. Also disclosed herein are methods of using such engineered enzymes for improving detection of target analyte proteins in an analytical assay.

### CROSS-REFERENCE TO RELATED APPLICATIONS

This application claims the benefit of and priority to U.S. Provisional Patent Application No. 63/395,986, filed Aug. 8, 2022. The entire disclosure of which is hereby incorporated by reference.

### REFERENCE TO A SEQUENCE LISTING XML

This application contains a Sequence Listing which has been submitted electronically in XML format. The Sequence Listing XML is incorporated herein by reference. Said XML file, created on Aug. 1, 2023, is named WAC-396US_SL.xml and is 12,004 bytes in size.

### BACKGROUND

Proteases are used for multiple analytical applications. For example, proteases are used in sequencing or peptide mapping of proteins as well as quality control testing of therapeutic proteins, such as monoclonal antibodies, antibody-drug conjugates (ADCs), and enzyme replacement therapies (ERTs). These analyses are frequently performed with liquid chromatography-mass spectrometry (LC-MS) instrumentation. Yet, problems exist in obtaining quality data when a protease acts on itself in via process called autolysis, which produces undesirable peptide byproducts of the protease that can obscure detection peaks of relevant protein analytes. This contaminates a sample with uninformative and disruptive peptides. Therefore, there exists a need for compositions and methods that minimize protease autolysis.

### SUMMARY OF THE DISCLOSURE

The present disclosure relates to recombinant protease enzymes, such as an endopeptidase Lys-C (Lys-C), Asp-N protease (Asp-N), and trypsin protease, containing one or more conservative amino acid substitutions that impart enhanced autolysis resistance to the enzymes. Also disclosed are methods of using such enzymes in analytical assays, such as liquid chromatography-mass spectrometry (LC-MS), among others, for improved detection and analysis of target protein analytes.

In an aspect, the disclosure provides a recombinant protease comprising one or more (e.g., 1, 2, 3, 4, 5, 6, 7, 8, 9, 10, 11, 12, 13, 14, 15, or more) conservative amino acid substitutions that enhance autolysis resistance of the protease as compared to the autolysis resistance of the protease in the absence of the one or more conservative substitutions, wherein the protease is Lys-C.

In an aspect, the disclosure provides a recombinant protease comprising one or more (e.g., 1, 2, 3, 4, 5, 6, 7, 8, 9, 10, 11, 12, 13, 14, 15, or more) conservative amino acid substitutions that enhance autolysis resistance of the protease as compared to the autolysis resistance of the protease in the absence of the one or more conservative substitutions, wherein the protease is Asp-N.

In an aspect, the disclosure provides a recombinant protease comprising one or more (e.g., 1, 2, 3, 4, 5, 6, 7, 8, 9, 10, 11, 12, 13, 14, 15, or more) conservative amino acid substitutions that enhance autolysis resistance of the protease as compared to the autolysis resistance of the protease in the absence of the one or more conservative substitutions, wherein the protease is trypsin.

In some embodiments, the one or more conservative substitutions comprise at least 1, at least 2, at least 3, at least 4, at least 5, or at least 6, at least 7, at least 8, at least 9, at least 10, at least 11, at least 12, at least 13, at least 14, or at least 15 conservative substitutions. In addition or alternatively, the one or more conservative amino acid substitutions comprise 4, 5, 6, 7, 8, 9, 10, 11, 12, 13, 14, or 15 conservative substitutions. In addition or alternatively, the one or more conservative substitutions is a lysine (Lys) to arginine (Arg) substitution. In addition or alternatively, the one or more conservative substitutions comprise a Lys to Arg substitution at one or more amino acid residues selected from residues 2, 39, 52, 54, 62, 104, 173, 178, 183, 205, 235, 254, 311, 360, and 408 of SEQ ID NO: 1 or residues 30, 49, 106, 155, and 203 of SEQ ID NO: 2. In addition or alternatively, the at least one or more conservative substitutions is an aspartate (Asp) to glutamate (Glu) substitution. In addition or alternatively, the one or more conservative substitutions comprise a Asp to Glu substitution at one or more amino acid residues selected from residues 2, 14, 46, and 130 of SEQ ID NO: 5. In addition or alternatively, the one or more conservative substitutions further comprise a Asp to Glu substitution at one or more amino acid residues 67 and 71 of SEQ ID NO: 5. In addition or alternatively, the one or more conservative substitutions is an Arg to Lys substitution. In addition or alternatively, the one or more conservative substitutions comprise an Arg to Lys substitution at one or more amino acid residues selected from residues 45, 49, 99, and 107 of SEQ ID NO: 8.

In some embodiments, the autolysis resistance of the protease is enhanced by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100% as compared to the autolysis resistance of the protease in the absence of the one or more conservative substitutions.

In some embodiments, the protease comprises one or more chemical modifications. In addition or alternatively, the one or more chemical modifications comprise an alkyl moiety, acetyl moiety, amide moiety, ester moiety, imine moiety, amidino moiety, guanidino moiety, or thioether moiety. In addition or alternatively, the alkyl moiety is selected from the group consisting of a methyl moiety, dimethyl moiety, octanal moiety, and cyclodextrin monoaldehyde moiety.

In some embodiments, the protease is porcine, bovine, rat, murine, avian, human, bacterial, fungal, or plant. In some embodiments, the protease is artificial or synthetic.

In some embodiments, an active site of the protease is free of amino acid substitutions.

In some embodiments, the protease comprises an amino acid sequence of SEQ ID NO: 3 or SEQ ID NO: 4. In addition or alternatively, the protease comprises an amino acid sequence of SEQ ID NO: 6 or SEQ ID NO: 7. In addition or alternatively, the protease comprises an amino acid sequence of SEQ ID NO: 9.

In an aspect, the disclosure provides a method of reducing a level of peptide byproducts of protease autolysis in an analytical assay, the method comprising the use of the recombinant protease of any one of the foregoing aspects and embodiments. In addition or alternatively, the analytical assay is selected from the group consisting of liquid chromatography (LC), LC-mass spectrometry (LC-MS), LC-UV, capillary electrophoresis (CE), gel electrophoresis (GE), and matrix-assisted laser desorption/ionization (MALDI). In addition or alternatively, the analytical assay is LC-MS.

In an aspect, the disclosure provides a polynucleotide (e.g., DNA or RNA) comprising a nucleic acid sequence encoding a recombinant protease of any of the foregoing aspects and embodiments. In some embodiments, the polynucleotide encodes a polypeptide having an amino acid of SEQ ID NO: 3 or SEQ ID NO: 4 or a variant thereof having at least 85% (e.g., at least 85%, 86%, 87%, 88%, 89%, 90%, 91%, 92%, 93%, 94%, 95%, 96%, 97%, 98%, 99%, or more) sequence identity to SEQ ID NO: 3 or SEQ ID NO: 4. In some embodiments, the polynucleotide encodes a polypeptide having an amino acid of SEQ ID NO: 6 or SEQ ID NO: 7 or a variant thereof having at least 85% (e.g., at least 85%, 86%, 87%, 88%, 89%, 90%, 91%, 92%, 93%, 94%, 95%, 96%, 97%, 98%, 99%, or more) sequence identity to SEQ ID NO: 6 or SEQ ID NO: 7. In some embodiments, the polynucleotide encodes a polypeptide having an amino acid of SEQ ID NO: X or a variant thereof having at least 85% (e.g., at least 85%, 86%, 87%, 88%, 89%, 90%, 91%, 92%, 93%, 94%, 95%, 96%, 97%, 98%, 99%, or more) sequence identity to SEQ ID NO: 9. In some embodiments, the polynucleotide further comprises a nucleic acid sequence encoding a protein tag (e.g., His-Tag, such as a 6×-His-Tag (SEQ ID NO: 10)).

In an aspect, the disclosure provides a nucleic acid expression vector comprising the polynucleotide of the foregoing aspect and embodiments. In some embodiments, the polynucleotide is operably linked to a promoter sequence (e.g., mammalian promoter, bacterial promoter, fungal promoter, or insect promoter). In some embodiments, the expression vector further comprises one or more (e.g., 1, 2, 3, 4, 5, 6, 7, 8, or more) regulatory sequences selected from the group consisting of a 5′ untranslated region (UTR), 3′ UTR, enhancer, insulator, intron, RNA export element, polyadenylation signal, and transcription terminator. In some embodiments, the expression vector is a plasmid. In some embodiments, the expression vector is a viral vector.

In an aspect, the disclosure provides a host cell comprising the recombinant protease, the polynucleotide, or the expression vector of any of the foregoing aspects and embodiments. In some embodiments, the host cell is a mammalian cell. In some embodiments, the host cell is a bacterial cell. In some embodiments, the host cell is a fungal cell. In some embodiments, the host cell is an insect cell. In some embodiments, the host is a plant cell.

In an aspect, the disclosure provides a kit comprising the recombinant protease, polynucleotide, expression vector, or host cell of any of the foregoing aspects and embodiments.

### Definitions

Unless defined otherwise, all technical and scientific terms used herein have the same meaning as is commonly understood by one of skill in the art to which the claimed subject matter pertains. Generally, nomenclatures utilized in connection with, and techniques of cell and tissue culture, molecular biology, and protein and polynucleotide chemistry described herein are those well-known and commonly used in the art. It is to be understood that the foregoing general description and the following detailed description are exemplary and explanatory only and are not restrictive of any subject matter claimed. The section headings used herein are for organizational purposes only and are not to be construed as limiting the subject matter described.

As used herein, the phrase “analytical assay” refers to any known assay used in the relevant art for the analysis of proteins. Non-limiting examples of analytical assays include those that are used for extraction, isolation, detection, sequence analysis, structure analysis, post-translational modification analysis, and assessment of the function of a target protein (‘target analyte’), among others. Specific examples of analytical assays include liquid chromatography (LC), LC-mass spectrometry (LC-MS), LC-UV, capillary electrophoresis (CE), gel electrophoresis (GE), matrix-assisted laser desorption/ionization (MALDI), hydrogen-deuterium exchange, protein sequencing, peptide mapping by electrophoresis, western blotting, protein nuclear magnetic resonance (NMR), protein footprinting, affinity purification, protein conformational studies, and proteomics, among others.

As used herein, the phrase “autolysis resistance” or variants thereof refers to a property of a recombinant protease enzyme described herein (e.g., Lys-C, Asp-N, and trypsin) to resist proteolytic cleavage via its own active site (i.e., self-cleavage). Protease enzymes are themselves proteins containing amino acid residues that can act as substrate residues for the protease's active site. Accordingly, the substitution of these residues with conservative amino acid residues that are not natural substrates for the enzyme's active site and/or chemical modification of said residues (e.g., alkylation) can enhance the resistance of the protease to self-cleavage. A recombinant protease enzyme disclosed herein can contain any number of amino acid residues that act as substrates for the enzyme's protease domain (e.g., 1, 2, 3, 4, 5, 6, or more amino acid residues). Accordingly, varying degrees of autolysis resistance can be conferred to the protease by conservative amino acid substitution at these residues. For example, autolysis resistance may be conferred to the protease by 1, 2, 3, 4, 5, 6, or more conservative amino acid substitutions. The enhancement in autolysis resistance can be by any amount, including, e.g., by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100% as compared to the autolysis resistance of the protease in the absence of the conservative substitutions. In some embodiments, the degree of protease autolysis resistance is proportional to the number of conservative amino acid substitutions (i.e., more conservative amino acid substitutions result in greater autolysis resistance).

As used herein, the phrase “chemical modification” refers to any process by which a molecule or macromolecule can be converted through a chemical reaction or a series of chemical reactions. Non-limiting examples of chemical modifications include addition of an alkyl moiety, acetyl moiety, amide moiety, ester moiety, imine moiety, amidino moiety, guanidino moiety, or thioether moiety. Non-limiting examples of an alkyl moiety include a methyl moiety, dimethyl moiety, octanal, and cyclodextrin monoaldehyde moiety.

As used herein, the terms “conservative substitution,” “conservative amino acid substitution,” and “conservative mutation” refer to a substitution of one or more amino acids for one or more different amino acids that exhibit similar physicochemical properties, such as polarity, electrostatic charge, and steric volume. These properties are summarized for each of the twenty naturally-occurring amino acids in Table 1 below.

TABLE 1
Representative physicochemical properties
of naturally-occurring amino acids

| | | | | Electrostatic | |
|---|---|---|---|---|---|
| | 3- | 1- | Side- | character at | |
| | Letter | Letter | chain | physiological | Steric |
| Amino Acid | Code | Code | polarity | pH (7.4) | volume† |
| Alanine | Ala | A | Nonpolar | Neutral | Small |
| Arginine | Arg | R | Polar | Cationic | Large |
| Asparagine | Asn | N | Polar | Neutral; | Intermediate |
| Aspartic acid | Asp | D | Polar | Anionic | Intermediate |
| Cysteine | Cys | C | Nonpolar | Neutral | Intermediate |
| Glutamic acid | Glu | E | Polar | Anionic | Intermediate |
| Glutamine | Gln | Q | Polar | Neutral | Intermediate |
| Glycine | Gly | G | Nonpolar | Neutral | Small |
| Histidine | His | H | Polar | Both neutral | Large |
| | | | | and cationic | |
| | | | | forms in | |
| | | | | equilibrium | |
| | | | | at pH 7.4 | |
| Isoleucine | Ile | I | Nonpolar | Neutral | Large |
| Leucine | Leu | L | Nonpolar | Neutral | Large |
| Lysine | Lys | K | Polar | Cationic | Large |
| Methionine | Met | M | Nonpolar | Neutral | Large |
| Phenylalanine | Phe | F | Nonpolar | Neutral | Large |
| Proline | Pro | P | Nonpolar | Neutral | Intermediate |
| Serine | Ser | S | Polar | Neutral | Small |
| Threonine | Thr | T | Polar | Neutral | Intermediate |
| Tryptophan | Trp | W | Nonpolar | Neutral | Bulky |
| Tyrosine | Tyr | Y | Polar | Neutral | Large |
| Valine | Val | V | Nonpolar | Neutral | Intermediate |

†based on a volume in A³: 50-100 is small, 100-150 is intermediate, 150-200 is large, and >200 is bulky

From this table it is appreciated that the conservative amino acid families include (i) G, A, V, L and I; (ii) D and E; (iii) C, S and T; (iv) H, K and R; (v) N and Q; and (vi) F, Y and W. A conservative mutation or substitution is therefore one that substitutes one amino acid for a member of the same amino acid family (e.g., a substitution of Ser for Thr or Lys for Arg).

As used herein, the phrase “peptide byproducts of protease autolysis” refers to fragments of a protease (e.g., Lys-C, Asp-N, or trypsin) produced by way of autolysis by the protease. Peptide byproducts of protease autolysis can be of various sizes, depending on the length of the protease amino acid sequence and the number of amino acid residues contained therein that can act as substrates for proteolytic cleavage by the active site of the protease. Such peptide byproducts are generally undesirable in the context of certain protein assays (e.g., analytical assays disclosed herein) as they may produce interference and negatively impact the sensitivity and/or specificity of measurements produced by these assays. Non-limiting lengths of peptide byproducts of proteolysis include peptides having at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 65, 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, or more amino acid residues.

“Percent (%) sequence identity” with respect to a reference polynucleotide or polypeptide sequence is defined as the percentage of nucleic acids or amino acids in a candidate sequence that are identical to the nucleic acids or amino acids in the reference polynucleotide or polypeptide sequence, after aligning the sequences and introducing gaps, if necessary, to achieve the maximum percent sequence identity. Alignment for purposes of determining percent nucleic acid or amino acid sequence identity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal alignment over the full length of the sequences being compared. For example, percent sequence identity values may be generated using the sequence comparison computer program BLAST. As an illustration, the percent sequence identity of a given nucleic acid or amino acid sequence, A, to, with, or against a given nucleic acid or amino acid sequence, B, (which can alternatively be phrased as a given nucleic acid or amino acid sequence, A that has a certain percent sequence identity to, with, or against a given nucleic acid or amino acid sequence, B) is calculated as follows:

100 multiplied by (the fraction X/Y)

where X is the number of nucleotides or amino acids scored as identical matches by a sequence alignment program (e.g., BLAST) in that program's alignment of A and B, and where Y is the total number of nucleic acids in B. It will be appreciated that where the length of nucleic acid or amino acid sequence A is not equal to the length of nucleic acid or amino acid sequence B, the percent sequence identity of A to B will not equal the percent sequence identity of B to A.

As used herein, the term “recombinant” refers to a protein encoded by a nucleic acid that has been cloned into an expression system capable of transcribing the gene for translation into a protein. Genetic modification of the recombinant nucleic acid can be used to produce mutant proteins, such as mutant proteases having one or more (e.g., 1, 2, 3, 4, 5, 6, or more) conservative amino acid substitutions.

As used herein, the term “vector” includes a nucleic acid vector, e.g., a DNA vector, such as a plasmid, a RNA vector, virus, or other suitable replicon (e.g., viral vector). A variety of vectors have been developed for the delivery of polynucleotides encoding exogenous proteins into a prokaryotic or eukaryotic cell. Expression vectors suitable for use with the compositions and methods described herein contain a polynucleotide sequence as well as, e.g., additional sequence elements used for the expression of proteins and, optionally, the integration of these polynucleotide sequences into the genome of a host cell. Certain vectors that can be used for the expression one or more (e.g., 1, 2, 3, or more) recombinant protease enzymes, as described herein, include plasmids that contain regulatory sequences, such as promoter and enhancer regions, which direct gene transcription. Other useful vectors for expression of protease enzymes contain polynucleotide sequences that enhance the rate of translation of these genes or improve the stability or nuclear export of the mRNA that results from gene transcription. These sequence elements may include, e.g., 5′ and 3′ untranslated regions (UTRs), an internal ribosomal entry site (IRES), and a polyadenylation signal site in order to direct efficient transcription of the gene carried on the expression vector. The expression vectors suitable for use with the compositions and methods described herein may also contain a polynucleotide encoding a marker for selection of cells that contain such a vector. Examples of a suitable marker are genes that encode resistance to antibiotics, such as ampicillin, chloramphenicol, kanamycin, nourseothricin, zeocin, neorseothricin, carbenicillin, tetracycline, streptomycin, and spectinomycin.

### DETAILED DESCRIPTION

The present disclosure features engineered protease enzymes, including endopeptidase Lys-C (Lys-C), Asp-N protease (Asp-N), and trypsin protease that exhibit enhanced autolysis resistance, i.e., resistance to self-cleavage. Enhanced autolysis resistance is produced by incorporating one or more (e.g., 1, 2, 3, 4, 5, 6, 7, 8, 9, 10, 11, 12, 13, 14, or 15 or more) conservative amino acid substitutions at specific amino acid residues of the protease, namely those residues that act as natural substrates for the protease's own active site. Such autolysis resistant proteases are useful for analytical assays in which minimization of interference by byproducts of protease autolysis is desirable, such as liquid chromatography-mass spectrometry (LC-MS), among others.

Protease autolysis creates interference and negatively impacts the sensitivity and specificity of LC-MS-based measurements. One issue encountered during peptide mapping, among others, is that protease enzymes are, in and of themselves, proteins and will self-digest (‘autolyze’) into peptide byproducts. Issues stemming from autolytic background peptides become more pronounced in cases where two or more proteases are used on the same sample. Digestion mixtures that are desired to have only peptide fragments from the protein analyte of interest (‘the target analyte’) will thus be contaminated with peptides from the protease(s) used in the sample preparation. Chromatographic peaks for these protease fragments appear during high-performance liquid chromatography (HPLC)-based separation, thus, making identification and structural characterization of the target analyte protein more difficult.

To date, there exists a need to minimize autolysis reaction byproducts in LC-MS analyses in order to reduce byproduct interference during detection of target analytes. Previous studies have suggested incorporation of non-conservative amino acid substitutions into a rodent trypsin protease to enhance autolysis resistance (Várallyay Biochem Biophys Res Commun. 243:56-60, 1998; incorporated by reference in its entirety); however, because non-conservative substitutions may allow for introduction of amino acids having different physicochemical properties from that of the substituted amino acid, such substitutions are generally undesirable, as they have the potential to disrupt the protein tertiary structure and, resultantly, its function. Here, the present disclosure provides protease enzymes genetically engineered to introduce one or more conservative amino acid substitutions, and, optionally, chemically or enzymatically alkylated for enhanced resistance to autolysis.

### Recombinant Autolysis-Resistant Proteases

### Lys-C

Lys-C (30 kDa) is a bacterial serine protease which hydrolyzes peptide bonds on the carboxyl side of lysine (Lys) residues, particularly Lys residues that are followed by proline residues. This enzyme generally produces peptide fragments that are long and have lower complexity. Lys-C exhibits optimal protease activity at a pH range of 7.0-9.0 and is highly resistant to strong denaturing conditions (e.g., high concentrations of urea). Lys-C is naturally found to occur in Lysobacter spp., including, e.g., Lysobacter enzymogenes (Jekel et al. Anal Biochem 134:347-54, 1983), Lysobacter antibioticus, Lysobacter sp. Root96, Lysobacter maris, as well as Shewanella spp., Aquimonas, Pseudofulvimonas, Tahibacter sp., Thalassocella spp., Achromobacter lyticus (M497-1) (Masaki et al. Agric Biol Chem 42:1443-5. 1978), and Myxobacteria Strain AL-1 (Wingard et al. J Bact 112:940-9, 1979). This protease is frequently used alone or in combination with other protease enzymes for various applications, including in-solution or in-gel protein digestion, phosphopeptide enrichment, protein mapping, peptide mass fingerprinting, mass spectrometry-based spectral matching, and proteomics.

The present disclosure provides mutated variants of a Lys-C protease that exhibit enhanced autolysis resistance. The disclosed Lys-C protease can be obtained or derived from any biological source, including bacteria and/or artificial expression or synthesis systems. In some embodiments, the Lys-C protease is obtained or derived from Achromobacter lyticus. In some embodiments, the Lys C-protease is obtained or derived from a Lysobacter spp. In some embodiments, the Lysobacter spp. is selected from the group consisting of Lysobacter enzymogenes, Lysobacter antibioticus, Lysobacter sp. Root96, and Lysobacter maris. In some embodiments, the Lys-C protease is obtained or derived from Myxobacteria Strain AL-1. In some embodiments, the Lys-C protease is obtained or derived from a Shewanella spp. In some embodiments, the Lys-C protease is obtained or derived from Aquimonas. In some embodiments, the Lys-C protease is obtained or derived from Pseudofulvimonas. In some embodiments, the Lys-C protease is obtained or derived from Tahibacter sp. In some embodiments, the Lys-C protease is obtained or derived from Thalassocella spp.

The wild-type amino acid sequence of Lys-C of Achromobacter lyticus is provided in SEQ ID NO: 1, below, with bold letters demarcating lysine (Lys; K) residues at amino acid positions 2, 39, 52, 54, 62, 104, 173, 178, 183, 205, 235, 254, 311, 360, and 408 that act as natural substrates for the enzyme's proteolytic active site.

(SEQ ID NO: 1)
MKRICGSLLLLGLSISAALAAPASRPAAFDYANLSSVDKVALRTMPAVDV
AKAKAEDLQRDKRGDIPRFALAIDVDMTPQNSGAWEYTADGQFAVWRQRV
RSEKALSLNFGFTDYYMPAGGRLLVYPATQAPAGDRGLISQYDASNNNSA
RQLWTAVVPGAEAVIEAVIPRDKVGEFKLRLTKVNHDYVGFGPLARRLAA
ASGEKGVSGSCNIDVVCPEGDGRRDIIRAVGAYSKSGTLACTGSLVNNTA
NDRKMYFLTAHHCGMGTASTAASIVVYWNYQNSTCRAPNTPASGANGDGS
MSQTQSGSTVKATYATSDFTLLELNNAANPAFNLFWAGWDRRDQNYPGAI
AIHHPNVAEKRISNSTSPTSFVAWGGGAGTTHLNVQWQPSGGVTEPGSSG
SPIYSPEKRVLGQLHGGPSSCSATGTNRSDQYGRVFTSWTGGGAAASRLS
DWLDPASTGAQFIDGLDSGGGTP

The Lys-C protease includes a protease domain that performs its enzymatic function. The amino acid sequence of the wild-type Lys-C protease domain is provided in SEQ ID NO: 2, below, with bold letters demarcating Lys residues at amino acid positions 30, 49, 106, 155, and 203 that act as natural substrates for the enzyme's proteolytic active site.

(SEQ ID NO: 2)
GVSGSCNIDVVCPEGDGRRDIIRAVGAYSKSGTLACTGSLVNNTANDRKM
YFLTAHHCGMGTASTAASIVVYWNYQNSTCRAPNTPASGANGDGSMSQTQ
SGSTVKATYATSDFTLLELNNAANPAFNLFWAGWDRRDQNYPGAIAIHHP
NVAEKRISNSTSPTSFVAWGGGAGTTHLNVQWQPSGGVTEPGSSGSPIYS
PEKRVLGQLHGGPSSCSATGTNRSDQYGRVFTSWTGGGAAASRLSDWLDP
ASTGAQFIDGLDSGGGTP

In some embodiments, the wild-type Lys-C enzyme is modified to incorporate one or more (e.g., 1, 2, 3, 4, 5, 6, 7, 8, 9, 10, 11, 12, 13, 14, 15, or more) conservative amino acid substitutions. In some embodiments, the wild-type Lys-C enzyme is modified to incorporate one conservative amino acid substitution. In some embodiments, the wild-type Lys-C enzyme is modified to incorporate two conservative amino acid substitutions. In some embodiments, the wild-type Lys-C enzyme is modified to incorporate three conservative amino acid substitutions. In some embodiments, the wild-type Lys-C enzyme is modified to incorporate four conservative amino acid substitutions. In some embodiments, the wild-type Lys-C enzyme is modified to incorporate five conservative amino acid substitutions. In some embodiments, the wild-type Lys-C enzyme is modified to incorporate six conservative amino acid substitutions. In some embodiments, the one or more conservative amino acid substitutions is a Lys to arginine (Arg) substitution. In some embodiments, the one or more conservative substitutions include a Lys to Arg substitution at one or more amino acid residues selected from residues 2, 39, 52, 54, 62, 104, 173, 178, 183, 205, 235, 254, 311, 360, and 408 of SEQ ID NO: 1, or combinations thereof, or residues 30, 49, 106, 155, and 203 of SEQ ID NO: 2, or combinations thereof. In some embodiments, the one or more conservative substitutions include a Lys to Arg substitution at amino acid residue 2 of SEQ ID NO: 1. In some embodiments, the one or more conservative substitutions include a Lys to Arg substitution at amino acid residue 39 of SEQ ID NO: 1. In some embodiments, the one or more conservative substitutions include a Lys to Arg substitution at amino acid residue 52 of SEQ ID NO: 1. In some embodiments, the one or more conservative substitutions include a Lys to Arg substitution at amino acid residue 54 of SEQ ID NO: 1. In some embodiments, the one or more conservative substitutions include a Lys to Arg substitution at amino acid residue 62 of SEQ ID NO: 1. In some embodiments, the one or more conservative substitutions include a Lys to Arg substitution at amino acid residue 104 of SEQ ID NO: 1. In some embodiments, the one or more conservative substitutions include a Lys to Arg substitution at amino acid residue 173 of SEQ ID NO: 1. In some embodiments, the one or more conservative substitutions include a Lys to Arg substitution at amino acid residue 178 of SEQ ID NO: 1. In some embodiments, the one or more conservative substitutions include a Lys to Arg substitution at amino acid residue 183 of SEQ ID NO: 1. In some embodiments, the one or more conservative substitutions include a Lys to Arg substitution at amino acid residue 205 of SEQ ID NO: 1. In some embodiments, the one or more conservative substitutions include a Lys to Arg substitution at amino acid residue 235 of SEQ ID NO: 1 or amino acid residue 30 of SEQ ID NO: 2. In addition or alternatively, the one or more conservative substitutions include a Lys to Arg substitution at amino acid residue 254 of SEQ ID NO: 1 or amino acid residue 49 of SEQ ID NO: 2. In addition or alternatively, the one or more conservative substitutions include a Lys to Arg substitution at amino acid residue 311 of SEQ ID NO: 1 or amino acid residue 106 of SEQ ID NO: 2. In addition or alternatively, the one or more conservative substitutions include a Lys to Arg substitution at amino acid residue 360 of SEQ ID NO: 1 or amino acid residue 155 of SEQ ID NO: 2. In addition or alternatively, the one or more conservative substitutions include a Lys to Arg substitution at amino acid residue 408 of SEQ ID NO: 1 or amino acid residue 203 of SEQ ID NO: 2. In some embodiments, wild-type Lys-C protease is modified with a Lys to Arg conservative substitution at amino acid residues 2, 39, 52, 54, 62, 104, 173, 178, 183, 205, 235, 254, 311, 360, and 408 of SEQ ID NO: 1 to produce a mutated Lys-C protease having an amino acid sequence of SEQ ID NO: 3, with bold letters demarcating Arg (substituted from Lys) residues at amino acid positions 2, 39, 52, 54, 62, 104, 173, 178, 183, 205, 235, 254, 311, 360, and 408 that individually and/or together confer enhanced autolysis resistance to the mutated Lys-C protease.

(SEQ ID NO: 3)
MRRICGSLLLLGLSISAALAAPASRPAAFDYANLSSVDRVALRTMPAVDV
ARARAEDLQRDRRGDIPRFALAIDVDMTPQNSGAWEYTADGQFAVWRQRV
RSERALSLNFGFTDYYMPAGGRLLVYPATQAPAGDRGLISQYDASNNNSA
RQLWTAVVPGAEAVIEAVIPRDRVGEFRLRLTRVNHDYVGFGPLARRLAA
ASGERGVSGSCNIDVVCPEGDGRRDIIRAVGAYSRSGTLACTGSLVNNTA
NDRRMYFLTAHHCGMGTASTAASIVVYWNYQNSTCRAPNTPASGANGDGS
MSQTQSGSTVRATYATSDFTLLELNNAANPAFNLFWAGWDRRDQNYPGAI
AIHHPNVAERRISNSTSPTSFVAWGGGAGTTHLNVQWQPSGGVTEPGSSG
SPIYSPERRVLGQLHGGPSSCSATGTNRSDQYGRVFTSWTGGGAAASRLS
DWLDPASTGAQFIDGLDSGGGTP

In some embodiments, wild-type Lys-C protease domain is modified with a Lys to Arg conservative substitution at amino acid residues 30, 49, 106, 155, and 203 of SEQ ID NO: 2 to produce a mutated Lys-C protease domain having an amino acid sequence of SEQ ID NO: 4, with bold letters demarcating Arg (substituted from Lys) residues at amino acid positions 30, 49, 106, 155, and 203 that individually and/or together confer enhanced autolysis resistance to the mutated Lys-C protease domain. Arg residues are not part of the active site of Lys-C. Therefore, these mutations are not expected to alter the activity of Lys-C.

(SEQ ID NO: 4)
GVSGSCNIDVVCPEGDGRRDIIRAVGAYSRSGTLACTGSLVNNTANDRRM
YFLTAHHCGMGTASTAASIVVYWNYQNSTCRAPNTPASGANGDGSMSQTQ
SGSTVRATYATSDFTLLELNNAANPAFNLFWAGWDRRDQNYPGAIAIHHP
NVAERRISNSTSPTSFVAWGGGAGTTHLNVQWQPSGGVTEPGSSGSPIYS
PERRVLGQLHGGPSSCSATGTNRSDQYGRVFTSWTGGGAAASRLSDWLDP
ASTGAQFIDGLDSGGGTP

In some embodiments, the Lys-C protease can be modified with one or more conservative amino acid residues described above such that the protease exhibits an enhancement in autolysis resistance by, e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100% as compared to the autolysis resistance of the Lys-C protease in the absence of the one or more conservative substitutions.

### Asp-N

Endoproteinase Asp-N (˜25 kDa; also known as flavastacin) is a zinc metalloprotease that specifically cleaves peptide bonds on the N-terminal side of aspartate (Asp; D) and cysteine (Cys; C) residues. Asp-N exhibits optimal protease activity at a pH range of 4.0-9.0 and is highly resistant to strong denaturing conditions (e.g., high concentrations of urea). Asp-N is commonly derived from a mutated Pseudomonas fragi strain (Ingrosso et al. Biochem Biophys Res Commun. 162:1528-34, 1989) and Stenotrophomonas maltophilia. This protease is frequently used alone or in combination with other protease enzymes for various applications, including in-solution or in-gel protein digestion, phosphopeptide enrichment, protein mapping, peptide mass fingerprinting, mass spectrometry-based spectral matching, and proteomics.

The present disclosure provides mutated variants of an Asp-N protease that exhibit enhanced autolysis resistance. The disclosed Asp-N protease can be obtained or derived from any biological source, including bacteria and/or artificial expression or synthesis systems. In some embodiments, the Asp-N protease is obtained or derived from Stenotrophomonas maltophilia.

The wild-type amino acid sequence of Asp-N derived from Stenotrophomonas maltophilia is provided in SEQ ID NO: 5, below, with bold letters demarcating aspartate (Asp; D) residues at amino acid positions 2, 14, 46, 67, 71, and 130 that act as natural substrates for the enzyme's proteolytic active site.

(SEQ ID NO: 5)
MDSIHASRNATAADVAVLIINNASSCGLALGIGSTAATAFAAVHWDCATG
YYSFAHEIGHLQGARHDIATDSSTSPYAYGHGYRYEPASGTGWRTIMAYN
CTRSCPRLNYWSNPNITYNGIPMGNANTADNQRVLVNTKHTVAGFR

In some embodiments, the wild-type Asp-N enzyme is modified to incorporate one or more (e.g., 1, 2, 3, 4, 5, 6, or more) conservative amino acid substitutions. In some embodiments, the wild-type Asp-N enzyme is modified to incorporate one conservative amino acid substitution. In some embodiments, the wild-type Asp-N enzyme is modified to incorporate two conservative amino acid substitutions. In some embodiments, the wild-type Asp-N enzyme is modified to incorporate three conservative amino acid substitutions. In some embodiments, the wild-type Asp-N enzyme is modified to incorporate four conservative amino acid substitutions. In some embodiments, the wild-type Asp-N enzyme is modified to incorporate five conservative amino acid substitutions. In some embodiments, the wild-type Asp-N enzyme is modified to incorporate six conservative amino acid substitutions. In some embodiments, the one or more conservative amino acid substitutions is an Asp to glutamate (Glu; E) substitution. In some embodiments, the one or more conservative substitutions include an Asp to Glu substitution at one or more amino acid residues selected from residues 2, 14, 46, 67, 71, and 130 of SEQ ID NO: 5, or combinations thereof. In some embodiments, the one or more conservative substitutions include an Asp to Glu substitution at amino acid residue 2 of SEQ ID NO: 5. In addition or alternatively, the one or more conservative substitutions include an Asp to Glu substitution at amino acid residue 14 of SEQ ID NO: 5. In addition or alternatively, the one or more conservative substitutions include an Asp to Glu substitution at amino acid residue 46 of SEQ ID NO: 5. In addition or alternatively, the one or more conservative substitutions include an Asp to Glu substitution at amino acid residue 67 of SEQ ID NO: 5. In addition or alternatively, the one or more conservative substitutions include an Asp to Glu substitution at amino acid residue 71 of SEQ ID NO: 5. In addition or alternatively, the one or more conservative substitutions include an Asp to Glu substitution at amino acid residue 130 of SEQ ID NO: 5. In addition or alternatively, wild-type Asp-N protease is modified with an Asp to Glu conservative substitution at amino acid residues 2, 14, 46, 67, 71, and 130 of SEQ ID NO: 5 to produce a mutated Asp-N protease having an amino acid sequence of SEQ ID NO: 6, with bold letters demarcating Glu (substituted from Asp) residues at amino acid positions 2, 14, 46, 67, 71, and 130 that individually and/or together confer enhanced autolysis resistance to the mutated Asp-N protease.

(SEQ ID NO: 6)
MESIHASRNATAAEVAVLIINNASSCGLALGIGSTAATAFAAVHWECATG
YYSFAHEIGHLQGARHEIATESSTSPYAYGHGYRYEPASGTGWRTIMAYN
CTRSCPRLNYWSNPNITYNGIPMGNANTAENQRVLVNTKHTVAGFR

In some embodiments, the wild-type Asp-N protease is not modified with one or more (e.g., 1, 2, or more) Asp to Glu conservative substitutions at the active site of the protease, encompassing amino acid positions 67 and 71 of SEQ ID NO: 5. Accordingly, the wild-type Asp-N protease is modified, in some embodiments, with an Asp to Glu conservative substitution at amino acid residues 2, 14, 46, and 130 of SEQ ID NO: 5 to produce a mutated Asp-N protease having an amino acid sequence of SEQ ID NO: 7, with bold letters demarcating Glu (substituted from Asp) residues at amino acid positions 2, 14, 46, and 130 that individually and/or together confer enhanced autolysis resistance to the mutated Asp-N protease. Bold and underlined letters demarcate the wild-type Asp residues within the active site of Asp-N (amino acid positions 67 and 71).

(SEQ ID NO: 7)
MESIHASRNATAAEVAVLIINNASSCGLALGIGSTAATAFAAVHWECATG
YYSFAHEIGHLQGARHDIATDSSTSPYAYGHGYRYEPASGTGWRTIMAYN
CTRSCPRLNYWSNPNITYNGIPMGNANTAENQRVLVNTKHTVAGFR

In some embodiments, the Asp-N protease can be modified with one or more conservative amino acid residues described above such that the protease exhibits an enhancement in autolysis resistance by, e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100% as compared to the autolysis resistance of the Asp-N protease in the absence of the one or more conservative substitutions.

### Trypsin

Trypsin (˜24 kDa) is a ubiquitously applied serine protease used for LC-MS-based peptide mapping and proteomics studies. This enzyme performs its proteolytic function by cleaving peptide chains at the carboxyl side of Lys or Arg residues. The enzyme is synthesized in the pancreas cells of vertebrates as an inactive precursor, trypsinogen, and subsequently converted into the active form by cleavage of the propeptide. The activation process can also proceed autocatalytically at physiological pH. Trypsin is mainly used for the tryptic cleavage of peptides into small sections for sequencing, detaching adherent cells from coated cell culture dishes, cleaving fusion proteins, activating zymogens (e.g., trypsinogen to trypsin), and for recombinant production of peptide hormones. Trypsin is also a component of some pharmaceutical preparations (e.g., ointments, dragees, and aerosols for inhalation).

Some efforts have been made to improve its analytical capabilities. Trypsin was originally sourced for analytical work from the purification of animal-derived materials. Porcine trypsin has become preferred for analytical applications, though bovine and human variants are also commercially available. Recombinantly-expressed and affinity-purified trypsin enzymes are now widely commercially available. Moreover, a practice to chemically alkylate (e.g., methylate) trypsin enzymes on their Lys residues has become a broadly accepted practice for reducing autolysis over the course of sample preparation. Nevertheless, trypsin sequences also contain Arg residues, which, just like Lys, are natural substrates for trypsin proteolysis. These Arg residues are be acted on by the trypsin active site, even by an amino-alkylated derivatized form of the enzyme.

The present disclosure provides mutated variants of a trypsin protease that exhibit enhanced autolysis resistance. The disclosed trypsin protease can be obtained or derived from any biological source, including bacterial and fungal (e.g., Streptomyces, E. coli, P. pastoris, H. Polymorpha, S. cerevisiae, and S. pombae), mammalian sources (e.g., bovine, porcine, avian, murine, rodent, and human), plant sources, and/or artificial expression or synthesis systems. In some embodiments, the trypsin is bovine trypsin. In some embodiments, the trypsin is porcine trypsin. In some embodiments, the trypsin is avian trypsin. In some embodiments, the trypsin is murine trypsin. In some embodiments, the trypsin is rodent trypsin. In some embodiments, the trypsin is human trypsin.

The wild-type amino acid sequence of porcine trypsin is provided in SEQ ID NO: 8, below, with bold letters demarcating Arg residues at amino acid positions 45, 49, 99, and 107 that act as natural substrates for the enzyme's proteolytic active site, and underlined letters corresponding to Lys residues at amino acid positions 89, 125, 139, 149, 170, 200, 202, and 208 that may be alkylated (e.g., methylated) to further enhance autolysis resistance.

(SEQ ID NO: 8)
IVGGYTCAANSIPYQVSLNSGSHFCGGSLINSQWVVSAAHCYKSRIQVRL
GEHNIDVLEGNEQFINAAKIITHPNFNGNTLDNDIMLIKLSSPATLNSRV
ATVSLPRSCAAAGTECLISGWGNTKSSGSSYPSLLQCLKAPVLSDSSCKS
SYPGQITGNMICVGFLEGGKDSCQGDSGGPVVCNGQLQGIVSWGYGCAQK
NKPGVYTKVCNYVNWIQQTIAAN

In some embodiments, the wild-type trypsin enzyme is modified to incorporate one or more (e.g., 1, 2, 3, 4, or more) conservative amino acid substitutions. In some embodiments, the wild-type trypsin enzyme is modified to incorporate one conservative amino acid substitution. In some embodiments, the wild-type trypsin enzyme is modified to incorporate two conservative amino acid substitutions. In some embodiments, the wild-type trypsin enzyme is modified to incorporate three conservative amino acid substitutions. In some embodiments, the wild-type trypsin enzyme is modified to incorporate four conservative amino acid substitutions. In some embodiments, the wild-type trypsin enzyme is modified to incorporate five conservative amino acid substitutions. In some embodiments, the wild-type trypsin enzyme is modified to incorporate six conservative amino acid substitutions. In some embodiments, the one or more conservative amino acid substitutions is an Arg to Lys substitution. In some embodiments, the one or more conservative substitutions include an Arg to Lys substitution at one or more amino acid residues selected from residues 45, 49, 99, and 107 of SEQ ID NO: 8, or combinations thereof. In some embodiments, the one or more conservative substitutions include an Arg to Lys substitution at amino acid residue 45 of SEQ ID NO: 8. In addition or alternatively, the one or more conservative substitutions include an Arg to Lys substitution at amino acid residue 49 of SEQ ID NO: 8. In addition or alternatively, the one or more conservative substitutions include an Arg to Lys substitution at amino acid residue 99 of SEQ ID NO: 8. In addition or alternatively, the one or more conservative substitutions include an Arg to Lys substitution at amino acid residue 107 of SEQ ID NO: 8. In addition or alternatively, wild-type trypsin protease is modified with an Arg to Lys conservative substitution at amino acid residues 45, 49, 99, and 107 of SEQ ID NO: 8 to produce a mutated trypsin protease having an amino acid sequence of SEQ ID NO: 9, with bold letters demarcating Lys (substituted from Arg) residues at amino acid positions 45, 49, 99, and 107 that individually and/or together confer enhanced autolysis resistance to the mutated trypsin protease, and underlined letters corresponding to Lys residues at amino acid positions 89, 125, 139, 149, 170, 200, 202, and 208 that may be alkylated (e.g., methylated) to further enhance autolysis resistance. Arg residues are not part of the active site of trypsin. Therefore, these mutations are not expected to alter the enzyme's proteolytic activity.

(SEQ ID NO: 9)
IVGGYTCAANSIPYQVSLNSGSHFCGGSLINSQWVVSAAHCYKSKIQVKL
GEHNIDVLEGNEQFINAAKIITHPNFNGNTLDNDIMLIKLSSPATLNSKV
ATVSLPKSCAAAGTECLISGWGNTKSSGSSYPSLLQCLKAPVLSDSSCKS
SYPGQITGNMICVGFLEGGKDSCQGDSGGPVVCNGQLQGIVSWGYGCAQK
NKPGVYTKVCNYVNWIQQTIAAN

In some embodiments, the wild-type trypsin protease can be modified with one or more conservative amino acid residues described above such that the protease exhibits an enhancement in autolysis resistance by, e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100% as compared to the autolysis resistance of the trypsin protease in the absence of the one or more conservative substitutions.

### Chemical Modification of Recombinant Proteases

Autolysis resistance of protease enzymes (e.g., Lys-C, Asp-N, and trypsin) genetically engineered to incorporate one or more (e.g., 1, 2, 3, 4, 5, 6, or more) conservative amino acid substitutions can be further enhanced by the addition of a chemical modification at one or more (e.g., 1, 2, 3, 4, 5, 6, or more) amino acid residues that act as natural substrates for the enzyme's active site or one or more amino acid residues that have been mutated via conservative amino acid substitution to enhance autolysis resistance. In some embodiments, the chemical modification is selected from the group consisting of an alkyl moiety, acetyl moiety, amide moiety, ester moiety, imine moiety, amidino moiety, guanidino moiety, or thioether moiety. For example, a well-known method for improving autolysis resistance of trypsin is addition of an alkyl group (e.g., mono-alkyl or di-alkyl) at Lys residues (Rice et al. Biochim Biophys Acta. 492:316-21, 1977; and Means et al. Biochem. 7:2192-2210, 1968).

Accordingly, the present disclosure provides compositions and methods for alkylation of recombinant proteases of the disclosure (e.g., Lys-C, Asp-N, and trypsin) in order to further improve autolysis resistance of the enzymes. The alkyl group may be attached to an amine group of an amino acid residue (e.g., Lys) of the enzyme. In some embodiments, the alkyl group may be a primary or branched C₁₋₁₂ alkyl group. In some embodiments, recombinant proteases of the present disclosure are those in which the alkyl group is a primary or branched C₁₋₄ alkyl group. Alkylation of protease enzymes is generally performed by reductive alkylation. The degree of alkylation of amino acid residues will depend on the reaction conditions of the reductive alkylation process. For example, if the reaction cycle is repeated a number of times and/or a higher reagent:enzyme ratio is used, then full alkylation, i.e., alkylation of all target residues will be achieved. In some embodiments, recombinant protease enzymes of the present disclosure may be fully di-alkylated at all of their target amino acid residues. In some embodiments, recombinant protease enzymes of the present disclosure may be partially alkylated at one or more (e.g., 1, 2, 3, 4, 5, 6, 7, 8, 9, 10, 11, 12, 13, 14, or 15 or more) of their target amino acid residues. In some embodiments, the alkyl moiety is selected from the group consisting of a methyl moiety, dimethyl moiety, octanal moiety, and cyclodextrin monoaldehyde moiety. An exemplary, non-limiting method for reductive methylation of trypsin is described herein in Example 2.

### Recombinant Protease Expression and Purification

### Recombinant Protease Expression

The recombinant protease enzymes disclosed herein (e.g., Lys-C, Asp-N, and trypsin) may be produced using a recombinant expression system. For example, a polynucleotide (e.g., DNA or RNA) encoding a mutated protease of the disclosure may be incorporated into a recombinant expression vector capable of supporting and facilitating the expression of the protease in a host cell.

In a non-limiting example, disclosed herein are methods for expressing an engineered protease of the disclosure using a recombinant expression system, including: (1) transforming a host cell with a recombinant nucleic acid comprising a sequence which encodes the mutated protease from an bacterial, fungal, plant, or mammalian source; and (2) culturing the host cell under conditions and for a time sufficient to allow for the stable expression of the protease; and (3) isolating the expression product from the culture medium.

Exemplary methods that can be used for effectuating the expression of one or more recombinant proteases of the disclosure in a host cell are described in further detail below. One platform that can be used to achieve effective intracellular concentrations of one or more proteases described herein in host cells is via stable expression of genes encoding these enzymes (e.g., by integration into the nuclear or mitochondrial genome of a host cell). These genes are polynucleotides that encode the primary amino acid sequence of the corresponding protein. In order to introduce such exogenous genes into a host cell, these genes can be incorporated into a vector. Vectors can be introduced into a cell by a variety of methods, including transformation, transfection, direct uptake, projectile bombardment, and by encapsulation of the vector in a liposome. Examples of suitable methods of transfecting or transforming cells are calcium phosphate precipitation, electroporation, microinjection, infection, lipofection, and direct uptake. Such methods are described in more detail, for example, in Green et al., Molecular Cloning: A Laboratory Manual, Fourth Edition (Cold Spring Harbor University Press, New York (2014)); and Ausubel et al., Current Protocols in Molecular Biology (John Wiley & Sons, New York (2015)), the disclosures of each of which are incorporated herein by reference.

Genes encoding therapeutic proteins of the disclosure can also be introduced into host cells by targeting a vector containing a gene encoding such an agent to cell membrane phospholipids.

Recognition and binding of the polynucleotide encoding one or more proteases of the disclosure by RNA polymerase is important for gene expression. As such, one may include sequence elements within the polynucleotide that exhibit a high affinity for transcription factors that recruit RNA polymerase and promote the assembly of the transcription complex at the transcription initiation site. Such sequence elements include, e.g., a promoter, the sequence of which can be recognized and bound by specific transcription initiation factors and ultimately RNA polymerase, and which is operably linked to (e.g., is upstream of) the protease coding sequence. General examples of promoter classes suitable for use with the disclosed compositions and methods include constitutive promoters, spatiotemporal promoters, inducible promoters, and synthetic promoters. Examples of suitable promoters for directing the transcription of a nucleic acid encoding one or more protease enzymes of the disclosure, particularly in a bacterial host, are the promoter of the lac operon of E. coli, the Streptomyces coelicolor agarase gene dagA promoters, the promoters of the Bacillus lichenformis α-amylase gene (amyL), the promoters of the Bacillus stearothermophilus maltogenic amylase gene (amyM), the promoters of the Bacillus Amyloliquefaciens α-amylase (amyQ), the promoters of the Bacillus subtilis xylA and xylB genes etc. For transcription in a fungal host, examples of useful promoters are those derived from the gene encoding A. oryzae TAKA amylase, Rhizomucor miehei aspartic proteinase, A. niger neutral α-amylase, A. niger acid stable α-amylase, A. niger glucoamylase, Rhizomucor miehei lipase, A. oryzae alkaline protease, A. oryzae triose phosphate isomerase, or A. nidulans acetamidase. Non-limiting examples of mammalian promoters include cytomegalovirus (CMV) promoter, CAG promoter, elongation factor 1α (EF1α) promoter, eukaryotic elongation factor 2 (EEF2) promoter, glyceraldehyde 3-phosphate dehydrogenase (GAPDH) promoter, phosphoglycerate kinase (PGK) promoter, actin promoter (e.g., CBA), CK8 promoter, TBG promoter, H1 promoter, 7SK promoter, and ubiquitin promoter. For transcription in a plant host, non-examples of useful promoters are a CaMV35S cauliflower mosaic virus promoter, actin promoter, ubiquitin (e.g., GMUBI3, GMUBI7, RUBQ2, RUBI3, ZMUBIl, UBB1, PVUBIl, and UBI7) promoter, tubulin promoter, eukaryotic initiation factor (EIF) promoter, ascorbate peroxidase (APX) promoter, phosphogluconate dehydrogenase (PGD1) promoter, R1G1 domain containing protein B (RIGIB) promoter, dehydration element binding (DREBla) promoter, salt tolerance zinc finger (STZ/ZAT10) promoter, wax production 1 (WXP1) promoter, hordein promoter, glutenin promoter, expansin promoter, ACC-oxidase promoter, E8 promoter, polygalacturonase (PG) promoter, dioscorin pDJ3S promoter, potato class I patatin promoter, GBSS-granule-bound starch synthase promoter, sporamin promoter, β-amylase, RA8 promoter, A9 promoter, TA29 promoter, OSNCED3 promoter, WSI18 promoter, R29A promoter, and OSPR10 promtoer. Various viral promoters and additional regulatory elements (e.g., enhancers, terminators, and the like) may also be used to direct the transcription of a nucleic acid encoding one or more protease enzymes of the disclosure, including, e.g., bacteriophage T7, T3 and SP6 promoter and/or terminator elements, among others.

Once a polynucleotide encoding one or more recombinant proteases of the disclosure has been internalized by the host cell extrachromosomally and/or incorporated into the nuclear DNA of the host cell, the transcription of this polynucleotide can be induced by methods known in the art. For example, expression can be induced by exposing the host cell to an external chemical reagent, such as an agent that modulates the binding of a transcription factor and/or RNA polymerase to the promoter and, thus, regulates gene expression. The chemical reagent can serve to facilitate the binding of RNA polymerase and/or transcription factors to the promoter, e.g., by removing a repressor protein that has bound the promoter. Alternatively, the chemical reagent can serve to enhance the affinity of the promoter for RNA polymerase and/or transcription factors such that the rate of transcription of the gene located downstream of the promoter is increased in the presence of the chemical reagent. Examples of chemical reagents that potentiate polynucleotide transcription by the above mechanisms are tetracycline and doxycycline. These reagents are commercially available (Life Technologies, Carlsbad, CA) and can be administered to a host cell in order to promote gene expression according to established protocols.

Other DNA sequence elements that may be included in polynucleotides for use in the compositions and methods described herein are enhancer sequences. Enhancers represent another class of regulatory elements that induce a conformational change in the polynucleotide containing the gene of interest such that the DNA adopts a three-dimensional orientation that is favorable for binding of transcription factors and RNA polymerase at the transcription initiation site. Thus, polynucleotides for use in the compositions and methods described herein include those that encode one or more protease enzymes of the disclosure and additionally include an enhancer sequence. Many enhancer sequences are now known from bacterial, avian, and mammalian genes. Non-limiting examples of are enhancers from genes that encode mammalian globin, elastase, albumin, α-fetoprotein, and insulin. Enhancers for use in the compositions and methods described herein also include those that are derived from the genetic material of a virus capable of infecting a eukaryotic cell. Examples are the SV40 enhancer on the late side of the replication origin (bp 100-270), the cytomegalovirus early promoter enhancer, the polyoma enhancer on the late side of the replication origin, and adenovirus enhancers. Additional enhancer sequences that induce activation of eukaryotic gene transcription are disclosed in Yaniv et al., Nature 297:17 (1982).

### Expression Vectors

A variety of vectors for the delivery of polynucleotides encoding exogenous proteins to the a host cell have been developed. Expression vectors for use in the compositions and methods described herein may contain one or more polynucleotides encoding one or more protease enzymes of the disclosure, and may further include, for example, one or more (e.g., 1, 2, 3, 4, 5, 6, 7, 8, 9, 10, or more) nucleic acid elements used to regulate the expression of these agents and/or the integration of such polynucleotides into the genome of a host cell.

In some embodiments, the vector may be an autonomously replicating vector, i.e., a vector which exists as an extrachromosomal entity, the replication of which is independent of chromosomal replication, e.g., a plasmid, bacteriophage, extrachromosomal element, mini-chromosome, or an artificial chromosome. Alternatively, the vector may be one which, when introduced into a host cell, is integrated into the host cell genome and replicated together with the chromosome(s) into which it has been integrated. Certain vectors that can be used for the expression of one or more engineered proteases described herein include plasmids that contain regulatory sequences, such as promoter and, optionally, enhancer regions, which direct gene transcription. Other useful vectors for expression of one or more protease enzymes of the disclosure contain polynucleotide sequences that enhance the rate of translation of these genes or improve the stability or nuclear export of the mRNA that results from gene transcription. These sequence elements include, e.g., 5′ and 3′ untranslated regions, an internal ribosome entry site (IRES), and polyadenylation signal site in order to direct efficient transcription of the gene carried on the expression vector. The expression vectors suitable for use with the compositions and methods described herein may also contain a polynucleotide encoding a marker for selection of cells that contain such a vector. Examples of a suitable marker are genes that encode resistance to antibiotics, such as ampicillin, chloramphenicol, kanamycin, and nourseothricin, among others.

In some embodiments, expression vectors of the present disclosure further include a polynucleotide encoding a protein tag, such as, a His-tag (e.g., 6×-His (SEQ ID NO: 10)), maltose binding protein tag, SNAP tag, FLAG tag, halotag, fluorescent protein tag, and the like.

Viral Vectors

Viral genomes provide a rich source of vectors that can be used for the efficient delivery of exogenous genes into a host cell. Viral genomes are particularly useful vectors for gene delivery as the polynucleotides contained within such genomes are typically incorporated into the nuclear genome of a host cell by generalized or specialized transduction. These processes occur as part of the natural viral replication cycle and do not require added proteins or reagents in order to induce gene integration. Examples of viral vectors are a retrovirus (e.g., Retroviridae family viral vector), adenovirus (e.g., Ad5, Ad26, Ad34, Ad35, and Ad48), parvovirus (e.g., adeno-associated viruses), coronavirus, negative strand RNA viruses such as orthomyxovirus (e.g., influenza virus), rhabdovirus (e.g., rabies and vesicular stomatitis virus), paramyxovirus (e.g. measles and Sendai), positive strand RNA viruses, such as picornavirus and alphavirus, and double stranded DNA viruses including adenovirus, herpesvirus (e.g., Herpes Simplex virus types 1 and 2, Epstein-Barr virus, and cytomegalovirus), and poxvirus (e.g., vaccinia, modified vaccinia Ankara (MVA), fowlpox and canarypox). Other viruses include Norwalk virus, togavirus, flavivirus, reoviruses, papovavirus, hepadnavirus, human papilloma virus, human foamy virus, and hepatitis virus, for example. Examples of retroviruses include, but are not limited to, avian leukosis-sarcoma, avian C-type viruses, mammalian C-type, B-type viruses, D-type viruses, oncoretroviruses, HTLV-BLV group, lentivirus, alpharetrovirus, gammaretrovirus, spumavirus. Other examples are murine leukemia viruses, murine sarcoma viruses, mouse mammary tumor virus, bovine leukemia virus, feline leukemia virus, feline sarcoma virus, avian leukemia virus, human T-cell leukemia virus, baboon endogenous virus, Gibbon ape leukemia virus, Mason Pfizer monkey virus, simian immunodeficiency virus, simian sarcoma virus, Rous sarcoma virus and lentiviruses. Plant viruses may also be suitable for use with the compositions and methods disclosed herein, including but not limited to double-stranded DNA viruses such as caulimovirus and badnavirus, single-stranded DNA viruses such as geminiviridae, and RNA viruses such as, reoviridae (e.g., phtoreovirus, fijivirus, and oryzavirus), partiviridae (e.g., alphacryptovirus and betacryptovirus), rhabdoviridae (e.g., cytorhabdovirus and nucleorhabdovirus), bunyaviridae (e.g., tospovirus), tenulvirus, sequiviridae (e.g., tombusviridae), dianthovirus, luteovirus, machlomovirus, marafivirus, necrovirus, sobemovirus, tymovirus, enamovirus, idaeovirus, bromoviridae (e.g., cucumovirus, bromovirus, ilavirus, alfamovirus), comoviridae (e.g., nepovirus), tobamovirus, tobravirus, hordeivirus, furovirus, potexvirus, capillovirus, trichovirus, carlavirus, potyviridae, and closterovirus. Other examples of vectors are described, for example, in U.S. Pat. No. 5,801,030 A, the disclosure of which is incorporated herein by reference in its entirety.

Methods for Delivery of Recombinant Nucleic Acids to Host Cells

Techniques that can be used to introduce a polynucleotide, such as polynucleotide encoding one or more of the recombinant proteases disclosed herein, into a host cell are well known in the art. For example, electroporation can be used to permeabilize host cells by the application of an electrostatic potential to the cell of interest. Host cells subjected to an external electric field in this manner are subsequently predisposed to the uptake of exogenous nucleic acids. A similar technique, Nucleofection™, utilizes an applied electric field in order to stimulate the uptake of exogenous polynucleotides into the nucleus of a eukaryotic cell.

Additional techniques useful for the transfection of target cells are the squeeze-poration methodology. This technique induces the rapid mechanical deformation of cells in order to stimulate the uptake of exogenous DNA through membranous pores that form in response to the applied stress. This technology is advantageous in that a vector is not required for delivery of nucleic acids into a cell, such as a target cell.

Lipofection represents another technique useful for transfection of target cells. This method involves the loading of nucleic acids into a liposome, which often presents cationic functional groups, such as quaternary or protonated amines, towards the liposome exterior. This leads to uptake of the exogenous nucleic acids, for example, by direct fusion of the liposome with the cell membrane or by endocytosis of the complex. Similar techniques that exploit ionic interactions with the cell membrane to provoke the uptake of foreign nucleic acids are contacting a cell with a cationic polymer-nucleic acid complex. Exemplary cationic molecules that associate with polynucleotides so as to impart a positive charge favorable for interaction with the cell membrane are activated dendrimers, polyethylenimine, and diethylaminoethyl (DEAE)-dextran. Magnetic beads are another tool that can be used to transfect target cells in a mild and efficient manner, as this methodology utilizes an applied magnetic field in order to direct the uptake of nucleic acids.

Another useful tool for inducing the uptake of exogenous nucleic acids by target cells is laserfection, also called optical transfection, a technique that involves exposing a cell to electromagnetic radiation of a particular wavelength in order to gently permeabilize the cells and allow polynucleotides to penetrate the cell membrane. The bioactivity of this technique is similar to, and in some cases found superior to, electroporation.

Impalefection is another technique that can be used to deliver genetic material to target cells. It relies on the use of nanomaterials, such as carbon nanofibers, carbon nanotubes, and nanowires. Needle-like nanostructures are synthesized perpendicular to the surface of a substrate. DNA containing the gene, intended for intracellular delivery, is attached to the nanostructure surface. A chip with arrays of these needles is then pressed against cells or tissue. Cells that are impaled by nanostructures can express the delivered gene(s).

Magnetofection can also be used to deliver nucleic acids to target cells. The magnetofection principle is to associate nucleic acids with cationic magnetic nanoparticles. The magnetic nanoparticles are made of iron oxide, which is fully biodegradable, and coated with specific cationic proprietary molecules varying upon the applications. Their association with the nucleic acid vectors is achieved by salt-induced colloidal aggregation and electrostatic interaction. The magnetic particles are then concentrated on the target cells by the influence of an external magnetic field generated by magnets.

Another useful tool for inducing the uptake of exogenous nucleic acids by target cells is sonoporation, a technique that involves the use of sound (typically ultrasonic frequencies) for modifying the permeability of the cell plasma membrane to permeabilize the cells and allow polynucleotides to penetrate the cell membrane.

Microvesicles represent another potential vehicle that can be used to modify the genome of a target cell according to the methods described herein. For example, microvesicles that have been induced by the co-overexpression of the glycoprotein VSV-G with, e.g., a genome-modifying protein, such as a nuclease, can be used to efficiently deliver proteins into a cell that subsequently catalyze the site-specific cleavage of an endogenous polynucleotide sequence so as to prepare the genome of the cell for the covalent incorporation of a polynucleotide of interest, such as a gene or regulatory sequence.

### Protease Purification

Subsequent to cell-based or cell-free expression of the disclosed protease enzymes using the methods disclosed herein, said protease enzymes are purified and isolated for use. A variety of well-known protein purification methods may be used in conjunction with the disclosed methods. In a cell-based expression system, purification generally begins with preparation of a crude extract containing a complex mixture of all proteins from the cell cytoplasm and various other macromolecules, cofactors, and nutrients. Crude extracts are prepared, in some embodiments, using chemical methods, enzymatic methods, sonication, or a French press. Subsequently, debris may be removed from the crude extract by centrifugation, and the supernatant containing the expressed proteases is retrieved. Various well-known methods may then be used to isolate the recombinant protein from the supernatant, including but not limited to, chromatographic methods (e.g., affinity chromatography, HPLC, SEC, and IEC), protein precipitation, and cation exchange and gel filtration. Confirmation of protein purity may be performed by well-known methods, including, e.g., HPLC, MS, SDS-PAGE, ELISA, Bradford assay, ultraviolet-visible spectroscopy, activity assays, dynamic light scattering, microfluidic diffusional sizing, sedimentation velocity methods, and immunoblotting.

### Host Cells

A host cell suitable for use with the disclosed compositions and methods can be a eukaryotic or prokaryotic host cell well-known and routinely used in the art for recombinant protein expression. The host cell of the disclosure either comprising a polynucleotide or an expression vector of the disclosure, as defined herein, is advantageously used as a host cell in the recombinant production of a protease of the disclosure. The cell of the disclosure may be a cell of a higher organism such as a mammal or an insect, but can also be a microbial cell, e.g., a bacterial or a fungal (including yeast, such as budding and brewing yeast) cell, or a plant cell, in some embodiments.

In some embodiments, the host cell is a mammalian cell. The mammalian cell may be, without limitation, a Chinese hamster ovary (CHO) cell, human embryonic kidney 293 (HEK293) cell, and HEK293T cell, among others. In some embodiments, the host cell is an insect cell (e.g., Spodoptera frugiperda, such as a SF9 cell).

Alternatively, the host cell is a bacterial cell. Examples of suitable bacterial cells are gram-positive bacteria such as Bacillus subtilis, Bacillus lichenformis, Bacillus lentus, Bacillus brevis, Bacillus stearothermophilus, Bacillus alkalophilus, Bacillus amyloliquefaciens, Bacillus coagulans, Bacillus circulans, Bacillus lautus, Bacillus megaterium, Bacillus thuringiensis, Streptomyces lividans, or Streptomyces murinus, or gram-negative bacteria such as E. coli. The transformation of the bacteria may for instance be effected by protoplast transformation or by using competent cells in a manner known per se. An exemplary method for recombinantly expressing a protease of the present disclosure in E. coli is provided in Example 1.

Alternatively, the host cell is a fungal cell. In some embodiments, the fungal cell is a yeast cell. In some embodiments, the yeast cell is selected from the group consisting of Pichia pastoris, Hansenula polymorpha, Saccharomyces cerevisiae, Schizosaccharomyces pombae.

Cell-Free Expression Systems

Alternatively, the recombinant proteases of the disclosure may be expressed in vitro using a cell-free expression system which facilitates production of a recombinant protein without the use of living cells. Generally, cell-free expression systems include a solution containing ingredients necessary to direct protein synthesis, such as a protein-encoding polynucleotide, ribosomes, tRNA, enzymes, co-factors, amino acids, etc. Non-limiting examples of cell-free expression systems include the NEBExpress® Cell-free E. coli Protein Synthesis System and PURExpress® In Vitro Protein Synthesis kit, among others.

In addition or alternatively, the recombinant proteases of the disclosure may be synthesized, e.g., using solid-phase peptide synthesis. Solid phase peptide synthesis is a process used to chemically synthesize peptides on solid supports. In solid phase peptide synthesis, an amino acid or peptide is bound, usually via the C-terminus, to a solid support. New amino acids are added to the bound amino acid or peptide via coupling reactions. Due to the possibility of unintended reactions, protection groups are typically used. To date, solid phase peptide synthesis has become standard practice for chemical peptide synthesis.

### Assays for Assessing Autolysis and Target Proteolysis

The present disclosure further provides assays that are suitable for assessing the autolysis resistance of recombinant protease enzymes disclosed herein (e.g., Lys-C, Asp-N, and trypsin) as well as the digestion efficiency of target proteins (‘target proteolysis’). In some embodiments, autolysis resistance of the recombinant proteases of the disclosure is assessed by way of HPLC. In some embodiments, autolysis resistance of the recombinant proteases of the disclosure is assessed by way of MS. In some embodiments, autolysis resistance of the recombinant proteases of the disclosure is assessed by way of size exclusion chromatography (SEC). In some embodiments, autolysis resistance of the recombinant proteases of the disclosure is assessed by way of HPLC, MS, SEC, HPLC-UV, or any combination thereof. An exemplary, non-limiting method for testing protease autolysis resistance is described herein in Example 3.

Certain combinations of conservative amino acid substitutions and/or chemical modifications to any one of the recombinant protease enzymes disclosed herein (e.g., Lys-C, Asp-N, and trypsin) may produce unexpected effects on the proteolytic activity of said enzymes. Accordingly, the present disclosure provides methods for assaying the impact of disclosed mutations and/or chemical modifications on the protease's enzymatic activity on a target protein. In some embodiments, proteolytic efficacy of the recombinant proteases of the disclosure on a target protein is assessed by way of MS. In some embodiments, proteolytic efficacy of the recombinant proteases of the disclosure on a target protein is assessed by way of size exclusion chromatography (SEC). In some embodiments, proteolytic efficacy of the recombinant proteases of the disclosure on a target protein is assessed by way of HPLC, MS, SEC, HPLC-UV, or any combination thereof. An exemplary, non-limiting method for testing proteolytic efficacy of a protease is described herein in Example 4.

### Methods of Use

The present disclosure provides methods for using the disclosed recombinant proteases in a variety of uses. As discussed above, protease enzymes having enhanced autolysis resistance are particularly useful for analytical methods for analyzing proteins, including IPLC and/or MS. Autolysis produces undesirable peptide fragments from the protease itself during target analyte proteolysis, resulting in interference peaks that appear during IPLC or MS separation, thereby obfuscating peptide peaks corresponding to the analyte of interest. Thus, an autolysis-resistant protease advantageously minimized such interference peaks and improves the sensitivity and specificity of IPLC and/or MS measurements.

Furthermore, the disclosed autolysis resistant protease enzymes are well-suited for use in a variety of other applications, including HPLC-UV, development of cell and tissue culture protocols, protein degradation, protein sequencing, dissociation of adherent cells, analysis of protein-protein interactions, capillary electrophoresis (CE), gel electrophoresis (GE), matrix-assisted laser desorption/ionization (MALDI), hydrogen-deuterium exchange, peptide mapping by electrophoresis, western blotting, protein nuclear magnetic resonance (NMR), protein footprinting, affinity purification, protein imaging, proteomic analysis, and protein conformational studies.

Additionally, the disclosed protease enzymes may be used in conjunction with methods for digestion and analysis of protein therapeutics, viral vector proteomes, and protein compositions of T cell and CAR-T cell therapies. For example, recombinant proteins are frequently used in biotherapeutic applications and are typically characterized for their properties and modifications (e.g., purity, amino acid sequence, post-translational modifications, mutations, etc.) using MS (see, e.g., Fung et al. Bioanalysis 8:847-56, 2016), as well as other techniques, including HPLC, SEC, and HPLC-UV. As discussed herein, such analytic techniques are highly sensitive to byproducts of autolysis and would, therefore, benefit from use of autolysis-resistant protease enzymes that minimize contamination of the analyte sample with irrelevant and disruptive peptide peaks.

### Kits

The compositions described herein can be provided in a kit for use in any practical application described herein. The compositions may include one or more of the recombinant protease enzymes disclosed herein in a suitable container means. In some embodiments, the container means is any suitable container which houses, e.g., a liquid or lyophilized composition including, but not limited to, a vial, test tube, ampoule, bottle, or syringe. A syringe holds any volume of liquid suitable for injection into a subject, including, but not limited to, 0.5 cc, 1 cc, 2 cc, 5 cc, 10 cc, or more. In some embodiments, such containers include injection and/or blow-molded plastic containers into which the desired vials are retained. In some embodiments, kits also include printed material for use of the materials in the kit. In some embodiments, such containers include injection and/or blow-molded plastic containers into which the desired vials are retained. In some embodiments, kits also include printed material for use of the materials in the kit. Additionally, in some embodiments, the preparations contain stabilizers to increase the shelf-life of the kits and include, e.g., bovine serum albumin (BSA). Where the compositions are lyophilized, the kit contains, in some embodiments, further preparations of solutions to reconstitute the lyophilized preparations. Acceptable reconstitution solutions are well known in the art and include, e.g., phosphate buffered saline (PBS).

The term “packaging material” refers to a physical structure housing the components of the kit. In some embodiments, the packaging material maintains the components sterile and is made of material commonly used for such purposes (e.g., paper, corrugated fiber, glass, plastic, foil, ampules, etc.). In some embodiments, the label or packaging insert includes appropriate written instructions (e.g., instructing the user of the kit to perform one or more methods disclosed herein). Kits, in some embodiments, additionally include labels or instructions for using the kit components in any method of the disclosure. In some embodiments, a kit includes a compound in a pack or dispenser together with instructions for administering the compound in a method described herein. The instructions are, in some embodiments, on “printed matter,” e.g., on paper or cardboard within or affixed to the kit, or on a label affixed to the kit or packaging material, or attached to a vial or tube containing a component of the kit. Instructions are additionally included on a computer readable medium, such as, e.g., CD-ROMs, DVDs, flash memory devices, solid state memory, magnetic disks and disk devices, magnetic tapes, cloud computing systems and services, and the like, in some embodiments. In some cases, the program and instructions are permanently, substantially permanently, semi-permanently, or non-transitorily encoded on the media.

### EXAMPLES

The following examples are put forth to provide those of ordinary skill in the art with a description of how the compositions and methods described herein may be used, made, and evaluated, and are intended to be purely exemplary of the disclosure and are not intended to limit the scope of what the inventors regard as their disclosure

### Example 1: Vector Transformation and Recombinant Protein Expression and Purification

Nucleotide sequence encoding each of Lys-C (e.g., SEQ ID NO: 2 or SEQ ID NO: 2), Asp-N (e.g., SEQ ID NO: 6), and trypsin enzymes (e.g., SEQ ID NO: 9) are cloned into protein expression vectors such as pET20 or pET21b that contain affinity tags (such as His-tag) through standard molecular cloning procedures. The recombinant plasmids are verified for their sequence accuracy through DNA sequencing methods and mobilized into a suitable strain of E. coli through electroporation or chemical based transformation. The recombinant protein is induced for its expression at an appropriate growth stage of the bacterial host. The expressed protein is purified by affinity chromatography and analyzed for its quality by denaturing polyacrylamide gels (SDS-PAGE) and/or liquid chromatography-mass spectrometry (LC-MS). The list of recombinant plasmids and proteins that are expressed are as follows:
(i) pET20b-LysC_KtoR mt: A recombinant plasmid capable of expressing an engineered LysC mutant (SEQ ID NO: 2) that is resistant to autolysis. Lys-C contains Lys→Arg mutations.
(ii) pET20b-AspN_DtoE mt: A recombinant plasmid capable of expressing an engineered AspN mutant (SEQ ID NO: 6) that is resistant to autolysis. Asp-N contains Asp→Glu mutations.
(iii) pET20b-Trp_RtoK mt: A recombinant plasmid capable of expressing a mutant form of porcine trypsin for subsequent reductive methylation of Lys residues to make it highly resistant to autolysis. Trypsin contains Arg→Lys mutations.

### Example 2: Alkylation of Protease Enzymes

Reductive methylation of trypsin: Following purification of the recombinant protein, trypsin constructs are methylated at lysine residues (Heissel et al. PLoS ONE 14:e0218374, 2019) to improve its autolysis resistance. The protein is diluted with triethylammonium bicarbonate buffer (pH 8.5; 50 mM) to 1 mg/mL and treated with 2.2 μL of 36% formaldehyde, and 20 μL of sodium cyanoborohydride (NaBH₃CN; 0.6 M) per mg of trypsin for 10 minutes at room temperature. This reaction procedure is, optionally, carried at higher pH to minimize autolysis during the alkylation procedure. Optionally, the reaction is also carried out in the presence of a trypsin inhibitor, such as benzamidine. Following reductive methylation, the trypsin is, optionally, purified on Benzamidine-Sepharose 4b beads. Briefly, the beads (100 L/mg trypsin) are pre-treated with 3 volumes of ammonium bicarbonate buffer (pH 8, 50 mM), and sedimented by centrifugation at 5° C. After discarding the supernatant, trypsin solution is allowed to bind to the beads in the presence of a loading buffer (e.g., saturated L-phenylalanine in 50 mM ammonium bicarbonate) for 30 minutes through gentle rotation-based mixing. Protein bound beads are sedimented and washed with 3 volumes of ammonium bicarbonate buffer (50 mM, 3×), and centrifuged to remove the supernatant. Methylated trypsin is eluted with 12 mM HCl by gentle rotation at room temperature. As an alternative to purification by Benzamidine Sepharose 4B sorbent, cold acetone precipitation and centrifugation are applied to purify the trypsin. Extent of derivatization is measured by LC-MS.

Chemical derivatization of acidic amino acid residues in Asp-N: Treatment of mutant LysC or AspN variants is not required. However, if usage of wild-type AspN is desired alone or in combination with one or more engineered protease enzymes of the disclosure, chemical derivatization of acidic amino acid substrate can be advantageous. These acidic residue targeting proteases can be derivatized with carbodiimide in the presence of an excess concentration of an amine, including but not limited to ethanolamine. In this way, carboxylate groups of acidic amino acid side chains are converted to neutral amides without causing any significant amounts of protein crosslinking (Graceffa et al. Arch Biochem Biophys. 297:46-51, 1992). Since, the amide-containing reaction products cannot be acted upon as substrate, autolysis is further minimized. The procedure involves treatment of purified protein solution in 40 mM NaCl, 5 mM MOPS, 0.2 mM EDTA, pH 7.5, 0.2 mM DTT and protease inhibitor mixture (0.25 mM PMSF, 0.75 mM benzamidine, 1 μg/mL leupeptin) with 0.2-0.5 M ethanolamine (pH 4.0, from 1-2 M stock), 30 mM Mes buffer (pH 5.5 from 0.5 M stock), and 12 mM 1-ethyl-3-(3-dimethylaminoropyl)carbodiimide (EDC)(from 100 mM stock) for 2-3 hours at room temperature and quenched with 0.1-0.2 M β-mercaptoethanol. Autolysis resistance of the obtained engineered protease is tested, as described in Example 3, below. Extents of derivatization are measured by LC-MS.

### Example 3: Autolysis Assays

Autodigestion of a purified, engineered protease is monitored by incubating defined amounts of the protease in 50 mM ammonium bicarbonate buffer at 37° C. for 10 minutes to 16 hours and analyzing the resulting solution by reverse-phase high-performance liquid chromatography (RP-HPLC) using gradient chromatography. Mobile phases for this analysis include 0.1% trifluoroacetic acid (TFA) acidified water (A) and acetonitrile (B). Difluoroacetic acid or formic acid is also applied as a mobile phase additive along with other types of RP columns. The percentage of intact protein and autolytic peptides is evaluated by LC-UV or LC-MS analysis. Alternatively, size exclusion chromatography can be applied to assay intact versus autolyzed protease.

### Example 4: Protocol to Test the Digestion Efficiency of an Engineered Protease

A target protein, such as NIST mAb reference material 8671 or a small protein (e.g., a lysozyme or cytochrome C)(10 μg in 10 μL) is denatured with 6 M guanidinium hydrochloride (90 μL), and treated with dithiotreitol (2 μL, 250 mM) for 30 minutes at room temperature to reduce the disulfide bonds. The reduced form of cysteines is be alkylated by iodoacetamide (3 L, 350 mM) in dark for 30 minutes at room temperature. Subsequently, the protein is desalted on a gel filtration by gravity or spin column using digestion buffer (100 mM Tris pH 7.5), concentration adjusted to 0.2 μg/L, and digested with protease (1:5 or 1:20 ratio) for 60 minutes at 37° C. The reaction is quenched with 1% formic acid (20 μL) and stored at −20° C. for subsequent LC-MS analysis.

Liquid chromatography is performed with an ACQUITY Premier Peptide CSH C18 column (130 Å, 1.7 μm, 2.1×150 mm) using a 65° C. column temperature and 0.1% formic acid or 0.05% difluoroacetic acid-modified water and acetonitrile mobile phase. A 0.25 mL/min flow rate is applied. Gradient conditions are programmed with a hold at 1% B solution for 5 minutes, change to 40% B solution in 65 minutes, change to 70% B solution in 3 minutes followed by a hold of 2 minutes, then a switch to re-equilibration conditions (1% B solution), and a hold for 15 minutes.

Mass spectrometry data is acquired in full-scan mode using a time of flight mass spectrometer operating with a scan range of 50-2000 m/z at 2 Hz in positive ion mode. Electrospray source conditions are programmed for a 350° C. desolvation temperature, 20V cone voltage, and 1.2 kV capillary voltage when using a BioAccord instrument containing a benchtop ToF mass spectrometer. Fragmentation data are acquired in MSe mode by ramping the cone voltage to 60V-120V range.

### Other Embodiments

Various modifications and variations of the described disclosure will be apparent to those skilled in the art without departing from the scope and spirit of the disclosure. Although the disclosure has been described in connection with specific embodiments, it should be understood that the disclosure as claimed should not be unduly limited to such specific embodiments. Indeed, various modifications of the described modes for carrying out the disclosure that are obvious to those skilled in the art are intended to be within the scope of the disclosure. Other embodiments are in the claims.

1. A recombinant protease comprising one or more conservative amino acid substitutions that enhance autolysis resistance of the protease as compared to the autolysis resistance of the protease in the absence of the one or more conservative substitutions, wherein the protease is endopeptidase Lys-C (Lys-C).

2. A recombinant protease comprising one or more conservative amino acid substitutions that enhance autolysis resistance of the protease as compared to the autolysis resistance of the protease in the absence of the one or more conservative substitutions, wherein the protease is Asp-N protease (Asp-N).

3. A recombinant protease comprising one or more conservative amino acid substitutions that enhance autolysis resistance of the trypsin enzyme as compared to the autolysis resistance of the trypsin enzyme in the absence of the one or more conservative substitutions, wherein the protease is trypsin.

4. The recombinant protease of claim 1, wherein the one or more conservative substitutions comprise at least 1, at least 2, at least 3, at least 4, at least 5, at least 6, at least 7, at least 8, at least 9, at least 10, at least 11, at least 12, at least 13, at least 14, or at least 15 conservative substitutions.

5. The recombinant protease of claim 4, wherein the one or more conservative amino acid substitutions comprise 4, 5, 6, 7, 8, 9, 10, 11, 12, 13, 14, or 15 conservative substitutions.

6. The recombinant protease of any one of claim 1, wherein the one or more conservative substitutions is a lysine (Lys) to arginine (Arg) substitution.

7. The recombinant protease of claim 6, wherein the one or more conservative substitutions comprise a Lys to Arg substitution at one or more amino acid residues selected from residues 2, 39, 52, 54, 62, 104, 173, 178, 183, 205, 235, 254, 311, 360, and 408 of SEQ ID NO: 1 or residues 30, 49, 106, 155, and 203 of SEQ ID NO: 2.

8. The recombinant protease of claim 2, wherein the at least one or more conservative substitutions is an aspartate (Asp) to glutamate (Glu) substitution.

9. The recombinant protease of claim 8, wherein the one or more conservative substitutions comprise an Asp to Glu substitution at one or more amino acid residues selected from residues 2, 14, 46, and 130 of SEQ ID NO: 5.

10. The recombinant protease of claim 9, wherein the one or more conservative substitutions further comprise an Asp to Glu substitution at one or more of amino acid residues 67 and 71 of SEQ ID NO: 5.

11. The recombinant protease of claim 3, wherein the one or more conservative substitutions is an Arg to Lys substitution.

12. The recombinant protease of claim 11, wherein the one or more conservative substitutions comprise an Arg to Lys substitution at one or more amino acid residues selected from residues 45, 49, 99, and 107 of SEQ ID NO: 9.

13. (canceled)

14. The recombinant protease of claim 1, wherein the protease comprises one or more chemical modifications.

15.-17. (canceled)

18. The recombinant protease of claim 1, wherein an active site of the protease is free of amino acid substitutions.

19. The recombinant protease of claim 1, wherein the protease comprises an amino acid sequence of SEQ ID NO: 2 or SEQ ID NO: 4.

20. The recombinant protease of claim 2, wherein the protease comprises an amino acid sequence of SEQ ID NO: 6 or SEQ ID NO: 7.

21. The recombinant protease of claim 3, wherein the protease comprises an amino acid sequence of SEQ ID NO: 9.

22. A method of reducing a level of peptide byproducts of protease autolysis in an analytical assay, the method comprising the use of the recombinant protease of claim 1.

23. The method of claim 22, wherein the analytical assay is selected from the group consisting of liquid chromatography (LC), LC-mass spectrometry (LC-MS), LC-UV, capillary electrophoresis (CE), gel electrophoresis (GE), and matrix-assisted laser desorption/ionization (MALDI).

24. The method of claim 23, wherein the analytical assay is LC-MS.


SEQUENCE LISTING (10 sequences)

SEQ ID NO: 1 - protein, 473 aa, Achromobacter lyticus
MKRICGSLLLLGLSISAALAAPASRPAAFDYANLSSVDKVALRTMPAVDVAKAKAEDLQRDKRGDIPRFALAIDVDMTPQNSGAWEYTADGQFAVWRQRVRSEKALSLNFGFTDYYMPAGGRLLVYPATQAPAGDRGLISQYDASNNNSARQLWTAVVPGAEAVIEAVIPRDKVGEFKLRLTKVNHDYVGFGPLARRLAAASGEKGVSGSCNIDVVCPEGDGRRDIIRAVGAYSKSGTLACTGSLVNNTANDRKMYFLTAHHCGMGTASTAASIVVYWNYQNSTCRAPNTPASGANGDGSMSQTQSGSTVKATYATSDFTLLELNNAANPAFNLFWAGWDRRDQNYPGAIAIHHPNVAEKRISNSTSPTSFVAWGGGAGTTHLNVQWQPSGGVTEPGSSGSPIYSPEKRVLGQLHGGPSSCSATGTNRSDQYGRVFTSWTGGGAAASRLSDWLDPASTGAQFIDGLDSGGGTP

SEQ ID NO: 2 - protein, 268 aa, Achromobacter lyticus
GVSGSCNIDVVCPEGDGRRDIIRAVGAYSKSGTLACTGSLVNNTANDRKMYFLTAHHCGMGTASTAASIVVYWNYQNSTCRAPNTPASGANGDGSMSQTQSGSTVKATYATSDFTLLELNNAANPAFNLFWAGWDRRDQNYPGAIAIHHPNVAEKRISNSTSPTSFVAWGGGAGTTHLNVQWQPSGGVTEPGSSGSPIYSPEKRVLGQLHGGPSSCSATGTNRSDQYGRVFTSWTGGGAAASRLSDWLDPASTGAQFIDGLDSGGGTP

SEQ ID NO: 3 - protein, 473 aa, synthetic construct
MRRICGSLLLLGLSISAALAAPASRPAAFDYANLSSVDRVALRTMPAVDVARARAEDLQRDRRGDIPRFALAIDVDMTPQNSGAWEYTADGQFAVWRQRVRSERALSLNFGFTDYYMPAGGRLLVYPATQAPAGDRGLISQYDASNNNSARQLWTAVVPGAEAVIEAVIPRDRVGEFRLRLTRVNHDYVGFGPLARRLAAASGERGVSGSCNIDVVCPEGDGRRDIIRAVGAYSRSGTLACTGSLVNNTANDRRMYFLTAHHCGMGTASTAASIVVYWNYQNSTCRAPNTPASGANGDGSMSQTQSGSTVRATYATSDFTLLELNNAANPAFNLFWAGWDRRDQNYPGAIAIHHPNVAERRISNSTSPTSFVAWGGGAGTTHLNVQWQPSGGVTEPGSSGSPIYSPERRVLGQLHGGPSSCSATGTNRSDQYGRVFTSWTGGGAAASRLSDWLDPASTGAQFIDGLDSGGGTP

SEQ ID NO: 4 - protein, 268 aa, synthetic construct
GVSGSCNIDVVCPEGDGRRDIIRAVGAYSRSGTLACTGSLVNNTANDRRMYFLTAHHCGMGTASTAASIVVYWNYQNSTCRAPNTPASGANGDGSMSQTQSGSTVRATYATSDFTLLELNNAANPAFNLFWAGWDRRDQNYPGAIAIHHPNVAERRISNSTSPTSFVAWGGGAGTTHLNVQWQPSGGVTEPGSSGSPIYSPERRVLGQLHGGPSSCSATGTNRSDQYGRVFTSWTGGGAAASRLSDWLDPASTGAQFIDGLDSGGGTP

SEQ ID NO: 5 - protein, 146 aa, Stenotrophomonas maltophilia
MDSIHASRNATAADVAVLIINNASSCGLALGIGSTAATAFAAVHWDCATGYYSFAHEIGHLQGARHDIATDSSTSPYAYGHGYRYEPASGTGWRTIMAYNCTRSCPRLNYWSNPNITYNGIPMGNANTADNQRVLVNTKHTVAGFR

SEQ ID NO: 6 - protein, 146 aa, synthetic construct
MESIHASRNATAAEVAVLIINNASSCGLALGIGSTAATAFAAVHWECATGYYSFAHEIGHLQGARHEIATESSTSPYAYGHGYRYEPASGTGWRTIMAYNCTRSCPRLNYWSNPNITYNGIPMGNANTAENQRVLVNTKHTVAGFR

SEQ ID NO: 7 - protein, 146 aa, synthetic construct
MESIHASRNATAAEVAVLIINNASSCGLALGIGSTAATAFAAVHWECATGYYSFAHEIGHLQGARHDIATDSSTSPYAYGHGYRYEPASGTGWRTIMAYNCTRSCPRLNYWSNPNITYNGIPMGNANTAENQRVLVNTKHTVAGFR

SEQ ID NO: 8 - protein, 223 aa, Sus sp. [sequence omitted]

SEQ ID NO: 9 - protein, 223 aa, synthetic construct
IVGGYTCAANSIPYQVSLNSGSHFCGGSLINSQWVVSAAHCYKSKIQVKLGEHNIDVLEGNEQFINAAKIITHPNFNGNTLDNDIMLIKLSSPATLNSKVATVSLPKSCAAAGTECLISGWGNTKSSGSSYPSLLQCLKAPVLSDSSCKSSYPGQITGNMICVGFLEGGKDSCQGDSGGPVVCNGQLQGIVSWGYGCAQKNKPGVYTKVCNYVNWIQQTIAAN

SEQ ID NO: 10 - protein, 6 aa, synthetic construct
HHHHHH